(19) United States
(12) Patent Application Publication
(10) Pub. No.: US 2024/0207541 A1
(43) Pub. Date: Jun. 27, 2024

(54) PORTABLE COMBINED INHALATION DEVICE SUITABLE FOR VIALS

(71) Applicants: RNAIMMUNE VACCINE (GUANGZHOU) CO., LTD., Guangdong (CN); SIRNAOMICS BIOPHARMACEUTICALS (GUANGZHOU) CO., LTD., Guangdong (CN); CHANGZHOU ZHENGYUAN MEDICAL TECHONLOGY CO., LTD., Jiangsu (CN)

(72) Inventors: Chun LU, Guangdong (CN); Shenggao TANG, Guangdong (CN); Yue WU, Guangdong (CN); Cheng WANG, Jiangsu (CN)

(73) Assignees: RNAIMMUNE VACCINE (GUANGZHOU) CO., LTD., Guangdong (CN); SIRNAOMICS BIOPHARMACEUTICALS (GUANGZHOU) CO., LTD., Guangdong (CN); CHANGZHOU ZHENGYUAN MEDICAL TECHONLOGY CO., LTD., Jiangsu (CN)

(21) Appl. No.: 18/288,761

(22) PCT Filed: Jun. 16, 2022
(86) PCT No.: PCT/CN2022/099085

§ 371 Date: Oct. 27, 2023

(30) Foreign Application Priority Data
Jun. 18, 2021 (CN) 202110678938.9

(51) Int. Cl. A61M 15/00; A61M 11/02

(52) U.S. Cl. CPC A61M 15/0036; A61M 11/02; A61M 15/0041

(57) ABSTRACT

The present invention relates to a portable combined inhalation device suitable for vials, comprising an atomizing device and a connecting device detachably connected to the atomizing device; the connecting device comprises a support on which an accommodating groove capable of accommodating a vial is formed, and a puncture device that is arranged on the support and can puncture a rubber stopper of the vial; the puncture device comprises a liquid channel and a gas channel. If the vial, the connecting device, and the atomizing device are connected, one end of the liquid channel communicates with the vial, and the other end communicates with the atomizing device; one end of the gas channel communicates with the vial, and the other end communicates with air. The present invention can directly introduce medicinal liquid in the vial into the atomizing device for atomization under the action of atmospheric pressure, without the need to first transfer the medicament in the vial to a dedicated atomizing bottle and then connect the dedicated atomizing bottle to the atomizing device, thereby being more convenient and safer to use, and greatly reducing the possibility of the medicinal liquid being contaminated; in addition, the device has a simple structure.

### TECHNICAL FIELD

The present invention belongs to the field of medical atomization equipment, and particularly relates to a portable combined inhalation device suitable for vials.

### BACKGROUND

Ampoules are widely used as inner packaging materials for sterile liquid preparations due to the characteristics of low cost, uniform material, high-temperature sterilization resistance, and the like. However, the ampoules are inconvenient for clinical use and pose a safety risk. For example, particles such as glass scraps and the like in the production process environment are easily inhaled through the instantaneous negative pressure at the ampoule filling and sealing bottleneck. There is a high risk for pathogenic bacteria in the hospital environment to be inhaled through the instantaneous negative pressure during use, causing contamination. Although plastic ampoules can reduce contamination in the production process to a certain extent, it is difficult to avoid environmental contamination during use. Therefore, in developed countries such as Europe and the United States, the trend of using vials instead of ampoules has become more and more significant in recent years.

Chinese patent CN111840715A discloses a combined portable inhalation device comprising a disposable dedicated bottle for an atomized medicament, which requires the use of the disposable dedicated bottle for the atomized medicament specified in the patent, and requires an additional production line for the disposable dedicated bottle for the atomized medicament, such that the cost is high; if other non-dedicated bottles are used to package medicinal liquid, the medicinal liquid needs to be taken out and then put into the disposable dedicated bottle for the atomized medicament for use, and the poor use environment or improper operations will lead to a high probability of cross-contamination.

### SUMMARY

The technical problems to be solved by the present invention are to overcome the defects in the prior art, such that the present invention provides a portable combined inhalation device suitable for vials.

In order to solve the technical problems described above, the present invention adopts the following technical schemes:

Provided is a portable combined inhalation device suitable for vials, the inhalation device comprising an atomizing device and a connecting device detachably connected to the atomizing device; the connecting device comprises a support on which an accommodating groove capable of accommodating a vial is formed, and a puncture device that is arranged on the support and can puncture a rubber stopper of the vial; the puncture device comprises a liquid channel and a gas channel. If the vial, the connecting device, and the atomizing device are connected, one end of the liquid channel communicates with the vial, and the other end communicates with the atomizing device; one end of the gas channel communicates with the vial, and the other end communicates with air.

After the connecting device is connected to the atomizing device, a plastic part on the lid of the vial containing medicinal liquid is removed to expose the rubber stopper, and the opening of the vial is mounted on the support in an inverted manner. The puncture device can puncture the rubber stopper and enter the vial, such that the medicinal liquid flows into the atomizing device from the liquid channel of the puncture device, and the gas channel can enable the pressure in the whole device system to be kept balanced and ensure the smooth flow of the medicinal liquid into the atomizing device.

Preferably, the support can support and fix the vial, or apply a clamping force to the vial, and preferably the accommodating groove of the support can be adapted to a vial with a lid diameter of 10-25 mm.

Preferably, the surface of the liquid channel of the connecting device, which is in direct contact with the medicinal liquid, is made of a specific inert material. This material has good compatibility with the medicinal liquid, and prevents the quality of the medicinal liquid from being affected by ion migration, mutual reaction, adsorption, and the like during use, so that the quality of the medicinal liquid is not affected during use.

Preferably, the connecting device is made of PP medical material, and the puncture device is made of 304 stainless steel.

Preferably, the connecting device is a disposable connecting device with individually-sealed sterile packaging.

Preferably, using a sterile material for packaging can ensure that the connecting device is sterile before use; the disposable connecting device is taken out of the packaging to avoid contamination when in contact with the medicinal liquid.

Preferably, the puncture device can puncture a stopper of an unpunctured liquid container without generating coring.

Preferably, the puncture device has a length of 2-30 mm, further preferably 2-20 mm, and still further preferably 3-15 mm.

In the present invention, the length of the puncture device refers to the distance from the bottom of the accommodating groove to the top end of the puncture device when the puncture device is positioned in the accommodating groove after the connecting device is connected to the atomizing device.

During practical production, the length of the puncture device can be set according to the size of the adapted vial. The puncture device is not preferable to be too long, or too short to ensure that the medicinal liquid can smoothly flow out and completely after the connecting device is in the butt joint with the vial.

Preferably, the liquid channel has a diameter of 0.4-7 mm, and further preferably 1-4 mm.

Preferably, the gas channel has a diameter of 0.4-7 mm, and further preferably 1-4 mm.

Preferably, an upper end of the liquid channel and an upper end of the gas channel are integrally formed as a sharp end of the puncture device, or the liquid channel and the gas channel are separately arranged. The upper end of the liquid channel and the upper end of the gas channel form a sharp end, respectively.

Preferably, the puncture device is fixedly connected to the support, extends upwards from the bottom, and is positioned in the accommodating groove.

Further preferably, the puncture device is sleeved with a detachable protective sleeve, such that the puncture device can be kept sterile before use, and the protective sleeve can prevent accidental injury to users.

Preferably, the puncture device is slidably connected to the support, and the puncture device is driven to pass through the bottom of the support and protrudes upwards into the accommodating groove while connecting the connecting device to the atomizing device. In this way, the puncture device does not need to be sleeved with a protective sleeve.

Further preferably, a mounting part is fixedly connected to the bottom of the support, and the mounting part is formed with a cavity; the atomizing device is provided with a fitting part fittingly connected to the mounting part; a position-limiting part is fixedly connected to the puncture device, and the position-limiting part can be fixedly connected to the fitting part; when the mounting part is fittingly connected to the fitting part, the puncture device can slide upwards relative to the support. Before use, a puncture part is positioned in the cavity of the mounting part; after the mounting part is fittingly connected to the fitting part, the puncture device enters the accommodating groove.

Still further preferably, the position-limiting part comprises a horizontal rod extending along a horizontal direction and a vertical rod extending along a vertical direction; a lower end portion of the vertical rod is fixedly connected to a middle portion of the horizontal rod, and an upper-end portion of the vertical rod is fixedly connected to a lower end portion of the puncture device; the vertical rod is slidably connected to the mounting part, and the horizontal rod is detachably connected to the fitting part; when the horizontal rod is fittingly connected to the fitting part, the position-limiting part can slide upwards relative to the support to drive the puncture device to slide upwards relative to the support.

Still further preferably, the vertical rod is of a hollow structure; if the connecting device is connected to the atomizing device, one end of the vertical rod communicates with the liquid channel, and the other end communicates with the atomizing device.

Preferably, an upper-end opening of the gas channel is higher than an upper-end opening of the liquid channel, and this arrangement can ensure that the medicinal liquid flows out smoothly and completely and can prevent gas from entering the liquid channel.

Preferably, if the connecting device is connected to the atomizing device, a gap for facilitating the communication of the gas channel with the air exists between the bottom of the support and the atomizing device.

Further preferably, the bottom of the support is provided with a position-limiting block; if the connecting device is connected to the atomizing device, a lower-end portion of the position-limiting block abuts against the atomizing device, thereby ensuring the existence of the gap between the connecting device and the atomizing device.

Preferably, the connecting device is detachably connected to the atomizing device through threads. This arrangement can ensure the stability of the assembled inhalation device and the tightness at the joint, and prevent the leakage of the medicinal liquid.

According to some embodiments, the atomizing device comprises a medicinal liquid channel, an atomizing sheet arranged in the medicinal liquid channel in an intercepting manner, and a motor; when the connecting device is connected to the atomizing device, the liquid channel communicates with the medicinal liquid channel.

Preferably, the surface of the medicinal liquid channel of the atomizing device, which is in direct contact with the medicinal liquid, is made of a specific inert material, and this material has good compatibility with the medicinal liquid and has no effect on the quality of the medicinal liquid during use.

According to some specific embodiments, the bottom of the support is fixedly connected to the mounting part, the atomizing device is provided with a fitting part fittingly connected to the mounting part, and the mounting part is arranged as a plug with an external thread formed on the outer surface; the fitting part is arranged as a socket, and the socket is a groove provided with an internal thread matching the external thread on the groove wall surface; the connecting device is detachably connected to the atomizing device through the external thread and the internal thread.

Preferably, the atomizing sheet is a piezoelectric ceramic atomizing sheet or a high-frequency ultrasonic atomizing sheet, and the atomizing sheet communicates with an electrode connector of a main unit through an electrode probe.

Preferably, the piezoelectric ceramic atomizing sheet adopts a microgrid structure, and the surface of the piezoelectric ceramic atomizing sheet comprises a plurality of micropores made by a laser; of course, the atomizing sheet can also be a high-frequency ultrasonic atomizing sheet, and the atomizing sheet extends along a longitudinal direction of a channel and divides the channel into two parts, wherein one part is a medicinal liquid channel, and the other part is an atomizing channel.

Further, the atomizing sheet assembly also comprises a vibrator that is arranged on an atomizing body or the atomizing sheet and can cause the atomizing sheet to vibrate.

The atomization of the medicinal liquid is achieved in the vibration process to improve the atomizing effect of the medicament.

Preferably, an included angle between the atomizing sheet and a central axis of the medicinal liquid channel is 0-90°, and the atomizing sheet is arranged in the medicinal liquid channel in an intercepting manner.

Further preferably, the atomizing sheet is intercepted in the medicinal liquid channel at an angle of 45° to the horizontal direction, which is convenient for cleaning the atomizing sheet from both sides of the atomizing sheet.

Preferably, the atomizing device is detachably assembled from a plurality of parts.

According to some specific embodiments of the present invention, the connecting device is arranged at an upper portion of the atomizing device, and the atomizing device comprises an atomizing body provided with a medicinal liquid channel, an atomizing sheet arranged in the medicinal liquid channel, and a main unit detachably connected to the bottom of the atomizing body from the top. By means of the disassembly and assembly among the three parts, the medicinal liquid in the vial can quickly flow from the medicinal liquid channel of the puncture device to the atomizing sheet through the medicinal liquid channel of the atomizing device, such that the atomization of the medicinal liquid is achieved. When the atomizing sheet needs to be cleaned, the atomizing body is directly detached to wash the atomizing sheet.

According to other specific and preferred embodiments of the present invention, the atomizing device comprises an atomizing body provided with a medicinal liquid channel, an atomizing sheet assembly detachably connected to the atomizing body, and a main unit detachably connected to the atomizing body; the atomizing sheet assembly comprises a medicinal liquid outflow channel communicating with the medicinal liquid channel of the atomizing body, and the medicinal liquid outflow channel is provided with an atomizing sheet in an intercepting manner; when the atomizing sheet needs to be cleaned, the atomizing sheet assembly is directly detached for washing.

Specifically, the atomizing sheet assembly and the atomizing body are connected in a snap-fit manner.

According to other specific and preferred embodiments of the present invention, the main unit and the atomizing body of the atomizing device are combined to form a socket, and the connecting device is detachably connected to the socket through a plug; the main unit and the atomizing body are also combined to form a medicinal liquid channel, the medicinal liquid channel communicates with the socket, and the atomizing sheet is arranged in the medicinal liquid channel in the atomizing body in an intercepting manner; when the atomizing sheet needs to be cleaned, the atomizing body is directly detached for washing.

Specifically, the atomizing body and the main unit are connected in a snap-fit manner; preferably, the main unit is provided with an unlock button, and after the unlock button is operated, a connector can be separated from an interface, such that the main unit and the atomizing body can be quickly detached.

Due to the implementation of the technical schemes described above, compared with the prior art, the present invention has the following advantages:

The present invention designs a portable combined inhalation device suitable for vials, having a connecting device capable of connecting the vial and an atomizing device; the inhalation device can directly introduce medicinal liquid in the vial into the atomizing device for atomization under the action of atmospheric pressure, without the need to first transfer the medicament in the vial to a dedicated atomizing bottle and then connect the dedicated atomizing bottle to the atomizing device, thereby being more convenient and safer to use, and greatly reducing the possibility of the medicinal liquid being contaminated; in addition, the device has a simple structure, parts of the device can be separately packaged, and the practicability is high.

### BRIEF DESCRIPTION OF THE DRAWINGS

The present invention will be further illustrated in detail with reference to the following accompanying drawings and specific embodiments.

FIG. 1 is a partial sectional view of a portable combined inhalation device suitable for vials of Example 1 (a protective sleeve is not shown);

FIG. 2 is an exploded view (partial sectional view) of the portable combined inhalation device suitable for vials of Example 1;

FIG. 3 is a schematic view (partial sectional view) of a mounting position of an atomizing sheet of a portable combined inhalation device suitable for vials of Example 2;

FIG. 4 is a schematic structural view (partial sectional view) of a puncture device of a portable combined inhalation device suitable for vials of Example 3;

FIG. 5 is a schematic view (partial sectional view) during the use of a portable combined inhalation device suitable for vials of Example 4;

FIG. 6 is a schematic view (partial sectional view) of an assembly method for a portable combined inhalation device suitable for vials of Example 5; and

FIG. 7 is a schematic view (partial sectional view) of another assembly method for a portable combined inhalation device suitable for vials of Example 6;
wherein: 1. a connecting device; 10. a support; 101. an accommodating groove; 11. a puncture device; 111. a liquid channel; 112. a gas channel; 113. a position-limiting part; 1131. a horizontal rod; 1132. a vertical rod; 12. a position-limiting block; 13. a mounting part; 14. a protective sleeve; 15. a medicinal liquid filter; 16. an air filter; 2. an atomizing device; 21. an atomizing body; 211. a medicinal liquid channel; 212. an atomizing sheet; 213. an atomizing nozzle; 214. a fitting part; 215. a connector; 216. an electrode probe; 22. a main unit; 23. an atomizing sheet assembly; 221. a switch button; 222. an unlock button; 223. an electrode connector; 224. an interface; 3. a vial.

### DETAILED DESCRIPTION

In order to make the aforementioned purposes, features, and advantages of the present application more comprehensible, the specific embodiments of the present application will be further illustrated in detail with reference to the accompanying drawings. In the following description, numerous specific details are set forth to facilitate a full understanding of the present application. The present application can, however, be implemented in many other ways different from those described herein, and it will be apparent to those skilled in the art that similar modifications can be made without departing from the spirit of the present application. Therefore, the present application is not limited by the specific embodiments disclosed below.

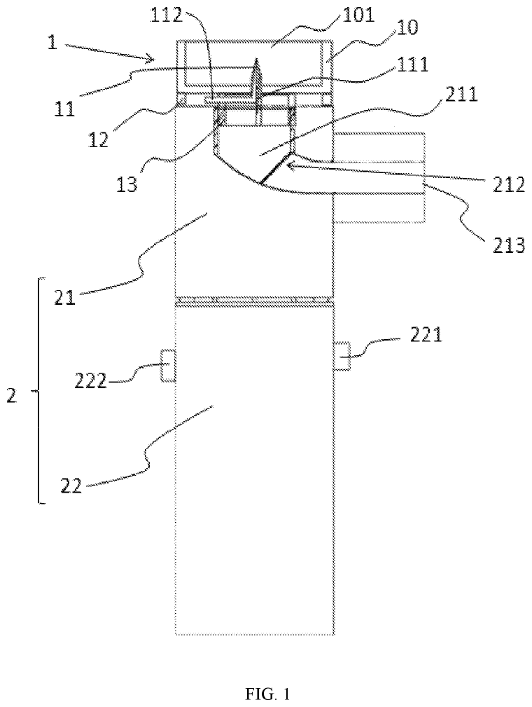

In the description of the present application, it should be understood that directions or positional relationships indicated by terms such as “longitudinal”, “upper”, “lower”, “left”, “right”, “vertical”, “horizontal”, “top”, “bottom”, “internal”, “external”, and the like are those shown based on FIG. 1, are merely intended to facilitate and simplify description rather than indicate or imply that the indicated device or element must have a specific direction and be structured and operated according to the specific direction, and should not be construed as limiting the present application.

In the present application, unless otherwise clearly specified and defined, a first feature “on” or “under” a second feature may refer to that the first feature and the second feature are in direct contact, or the first feature and the second feature are in indirect contact via an intermediate. Also, a first feature “on”, “above”, and “over” a second feature may be the first feature directly on or obliquely above the second feature, or simply mean that the first feature is at a higher level than the second feature. A first feature “under”, “beneath”, and “below” a second feature may be the first feature directly under or obliquely under the second feature, or simply means that the first feature is at a lower level than the second feature.

### Example 1

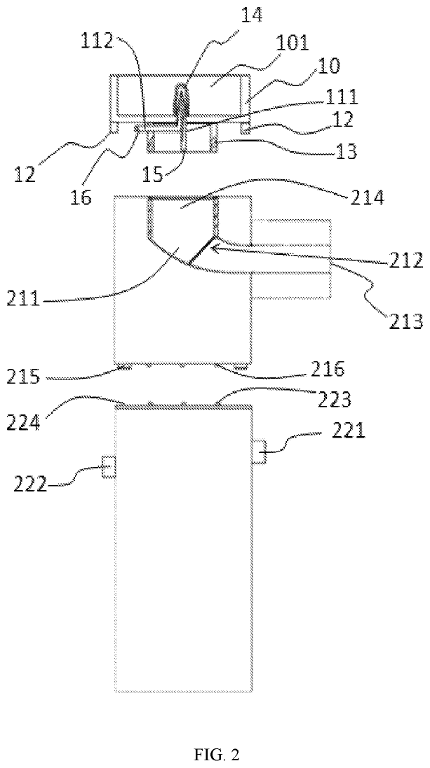

As shown in FIGS. 1 and 2, a portable combined inhalation device suitable for vials of this example comprises a connecting device 1 and an atomizing device 2, which are separately arranged and detachably connected.

As shown in FIGS. 1 and 2, the connecting device 1 comprises a puncture device 11 and a support 10.

Specifically, the support 10 has an accommodating groove 101 with an open upper end, the shape of the accommodating groove 101 matches the top of a vial, and the vial can be placed in the accommodating groove 101 in an inverted manner. The support 10 can be adapted according to the lids of vials 3 with different sizes to meet different requirements.

The puncture device 11 is arranged in the accommodating groove 101 of the support 10 and extends upwards from the middle portion of the bottom surface of the support 10, and the height of the puncture device 11 protruding upwards does not exceed a top end of the side wall of the support 10; an upper-end portion of the puncture device 11 forms a sharp end, which can puncture a rubber stopper of a lid of the vial 3; the puncture device 11 is provided with a gas channel 112 and a liquid channel 111; when the vial 3 is mounted on the support 10 in an opening direction, the puncture device 11 punctures the rubber stopper of the vial lid and enters the vial 3, and medicinal liquid in the vial 3 flows from the liquid channel 111 of the puncture device 11 into the atomizing device 2.

In this example, the length of the puncture device 11 extending upwards is 6 mm, and the length of the puncture device 11 puncturing and entering the vial 3 will affect the outflow or complete outflow of the medicinal liquid, and thus it is not preferable to be too long, nor too short.

In this example, the adapted vial 3 has a size of 8 mL, the liquid channel 111 has a diameter of 2 mm, and the gas channel 112 has a diameter of 2 mm.

In other examples, the length of the puncture device 11 and the diameters of the liquid and gas channels can be set according to the size of the adapted vial 3. Preferably, the puncture device 11 has a length of 2-30 mm, the liquid channel 111 has a diameter of 0.4-7 mm, and the gas channel 112 has a 0.4-7 mm diameter.

In this example, an upper end of the liquid channel 111 and an upper end of the gas channel 112 are integrally formed as the sharp end of the puncture device 11.

Specifically, an upper-end opening of the gas channel 112 is higher than an upper-end opening of the liquid channel 111, and this arrangement can ensure that the medicinal liquid flows out smoothly and completely, and can prevent gas from entering the liquid channel 111.

Specifically, an air inlet of the gas channel 112 is positioned at the outer side of the bottom of the support 10 and always communicates with the external atmospheric pressure, such that the pressure in the device system can be kept balanced, and the medicinal liquid in the vial 3 can smoothly flow into the atomizing device 2 for atomization. An outlet of the liquid channel 111 can be provided with a medicinal liquid filter 15 as required, and in this example, the medicinal liquid filter 15 has a pore size of 15 m. The air inlet can be provided with an air filter 16 to prevent environmental microorganisms from entering the vial and contaminating the medicinal liquid.

Specifically, the bottom of the support 10 is fixedly provided with a position-limiting block 12, which is used to support the connecting device 1 to stand on the atomizing device 2, and to enable a certain gap communicating with the outside to be formed between the bottom of the support 10 and the top of the atomizing device 2; a part of the channel 112 and the air inlet thereof are positioned in the gap, which can ensure that the air inlet of the channel 112 always communicates with the atmospheric pressure, to keep the pressure in the device system balanced.

Specifically, a mounting part 13 is fixedly connected to the bottom of the support 10, and the atomizing device 2 is provided with a fitting part 214 connected to the mounting part 13 fittingly. In this example, the mounting part 13 is arranged as a plug with an external thread formed on the outer surface; the fitting part 214 is arranged as a socket, and the socket is a groove provided with an internal thread matching the external thread on the groove wall surface; the connecting device 1 is detachably connected to the atomizing device 2 through the external thread and the internal thread.

Specifically, as shown in FIG. 2, the exterior of the puncture device 11 is sleeved with a detachable protective sleeve 14, such that the puncture device 11 can be kept sterile before use, and the protective sleeve 14 can prevent accidental injury to users, and be removed from the puncture device 11 before use.

The connecting device 1 of this example is a disposable vial connecting device, wherein the part of a medicinal liquid channel 211 in direct contact with the medicinal liquid should be made of a material with good compatibility, to prevent the quality of the medicinal liquid from being affected by ion migration, mutual reaction, adsorption, and the like during use.

The connecting device 1 of this example should be packaged with sterile material to ensure its sterile condition. The packaging material is disassembled before use, and the atomizing device 2 is started to achieve the atomization of the medicinal liquid in the vial 3 by means of connecting the connecting device to the atomizing device 2 and the vial 3.

As shown in FIG. 2, the atomizing device 2 comprises a medicinal liquid channel 211, an atomizing sheet 212 arranged in the medicinal liquid channel 211 in an intercepting manner, and a motor; when the connecting device 1 is connected to the atomizing device 2, the liquid channel 111 communicates with the medicinal liquid channel 211.

Specifically, the socket is arranged at an upper portion of an atomizing body 21, and the medicinal liquid channel 211 extends from the socket to an atomizing nozzle 213.

The medicinal liquid in the vial enters the socket at the upper portion of the atomizing body 21 through the liquid channel 111 of the connecting device 1, flows to the atomizing sheet 212 along the medicinal liquid channel 211 for the atomization of the medicinal liquid, and then outputs from the atomizing nozzle 213.

In this example, the socket is a groove provided with an internal thread on the groove wall surface, and the internal thread matches the external thread of the plug of the connecting device 1.

In this example, the atomizing sheet 212 is a piezoelectric ceramic atomizing sheet, and the atomizing sheet 212 communicates with a motor connector 223 of a main unit 22 through an electrode probe 216.

In this example, the piezoelectric ceramic atomizing sheet adopts a microgrid structure, and the surface of the piezoelectric ceramic atomizing sheet comprises a plurality of micropores made by a laser; of course, the atomizing sheet 212 can also be a high-frequency ultrasonic atomizing sheet; a channel in an atomizing sheet assembly 23 is divided into two parts, and the middle is separated by the atomizing sheet extending along a longitudinal direction of the channel, wherein one half is a medicinal liquid channel, and the other half is an atomizing channel.

The electrode probe 216 penetrates out from the bottom of the atomizing body 21, and the bottom surface is flush; preferably, the atomizing sheet 212 is intercepted in the medicinal liquid channel 211 at an angle of 0-90° to a horizontal direction. In this example, the atomizing sheet 212 is intercepted in the medicinal liquid channel 211 at an angle of 45° to the horizontal direction, which is convenient for cleaning the atomizing sheet 212 from both sides.

In this example, there are three electrode probes 216, one of which is used to detect signals such as temperature and pressure, and the remaining two of which are in circuit communication with the motor connector 223 at the top of the main unit 22, to form an atomization closed-loop circuit, and thus form the complete atomization main unit 22.

In this example, the atomizing body 21 is further provided with a vibrator, and the vibrator is specifically arranged on the atomizing sheet 212, such that the atomizing sheet 212 itself vibrates while performing the atomization, thereby improving the atomizing effect of the medicament.

The main unit 22 is detachably arranged at a lower portion of the atomizing body 21. The bottom of the atomizing body 21 is provided with a connector 215, the top of the main unit 22 is provided with an interface 224, and the atomizing body 21 and the main unit 22 are in an assembling or separating state under the fit or separation of the connector 215 and the interface 224.

Specifically, the main unit 22 is provided with a switch button 221 and an unlock button 222, wherein the connector 215 can be separated from the interface 224 after the unlock button 222 is operated, such that the main unit 22 and the atomizing body 21 can be quickly detached.

The common method adopts a spring clasp mode. Certainly, it can also adopt an electromagnet attraction and separation mode when the power is on or off, wherein a normal attraction is performed when the power is on. A normal separation is performed when the power is off.

Meanwhile, to ensure the accurate connection of the circuit, in this example, the motor connector 223 can be elastically and telescopically arranged on the main unit 22.

In summary, in this example, during clinical use, the atomizing body 21 after being cleaned and disinfected is first in butt joint with the main unit 22 to form the atomizing main unit 22, at this time, the electrode probe 216 is in a point-to-point connection to the motor connector 223 to form a closed-loop circuit, and the atomizing sheet 212 is connected to a power supply of the main unit 22, such that the atomization process can be started at any time; then, the plug of the connecting device 1 is aligned with the internally threaded groove of the socket on the atomizing body 21, and the connecting device 1 is screwed into the socket in a rotating manner; after the protective sleeve 14 of the puncture device 11 is removed, a plastic part on the lid of the vial 3 containing the medicinal liquid is removed to expose the rubber stopper, the opening of the vial is mounted in the support 10 of the connecting device 1 in an inverted manner, the puncture device 11 punctures the rubber stopper, and the medicinal liquid flows from the liquid channel 111 of the puncture device 11 to the atomizing sheet 212 along the medicinal liquid channel 211 through the socket; finally, the power switch is pressed or turned on, and the power drives the grid type atomizing sheet 212 to vibrate, such that the high-efficiency atomization of the medicinal liquid is achieved.

### Example 2

This example is another embodiment of a portable combined inhalation device suitable for vials, comprising the connecting device 1 and the atomizing device 2 which are separately arranged and detachably connected.

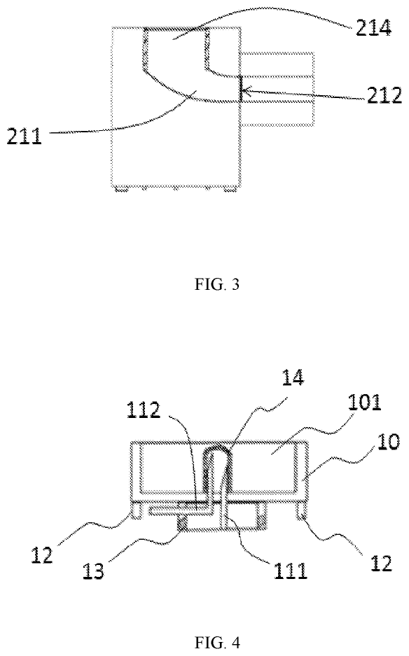

The portable combined inhalation device suitable for vials of this example is substantially the same as that of Example 1, except for the arrangement of the atomizing sheet 212. Specifically, as shown in FIG. 3, the atomizing sheet 212 is arranged in the medicinal liquid channel 211 in an intercepting manner perpendicular to a horizontal plane.

### Example 3

This example is still another embodiment of a portable combined inhalation device suitable for vials, comprising the connecting device 1 and the atomizing device 2 which are separately arranged and detachably connected.

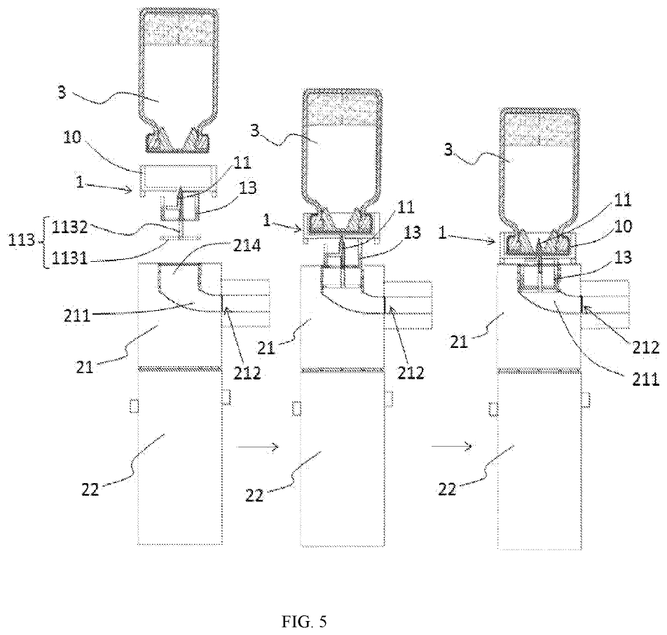

The portable combined inhalation device suitable for vials of this example is substantially the same as that of Example 1, except for the arrangement of the puncture device 11. Specifically, as shown in FIG. 4, the liquid channel 111 and the gas channel 112 are separately arranged, and the upper end of the liquid channel 111 and the upper end of the gas channel 112 form a sharp end, respectively; the upper-end opening of the gas channel 112 is higher than the upper-end opening of the liquid channel 111.

During use, after the connecting device 1 is connected to the socket of the atomizing body 21 through the plug, the plastic part on the lid of the vial 3 containing the medicinal liquid is removed to expose the rubber stopper, the opening of the vial is mounted in the support 10 of the connecting device 1 in an inverted manner, and the upper end of the liquid channel 111 and the upper end of the gas channel 112 puncture the rubber stopper, respectively; the medicinal liquid flows from the liquid channel 111 to the atomizing sheet 212 along the medicinal liquid channel 211 through the socket, and the gas channel 112 enables the pressure in the device system to keep balanced.

### Example 4

This example is yet another embodiment of a portable combined inhalation device suitable for vials, comprising the connecting device 1 and the atomizing device 2 which are separately arranged and detachably connected.

The portable combined inhalation device suitable for vials of this example is substantially the same as that of Example 1, except for the arrangement of the connecting device 1. The puncture device 11 is slidably connected to the support 10, and the puncture device 11 is driven to pass through the bottom of the support 10 and protrude upwards into the accommodating groove 101 while connecting the connecting device 1 and the atomizing device 2.

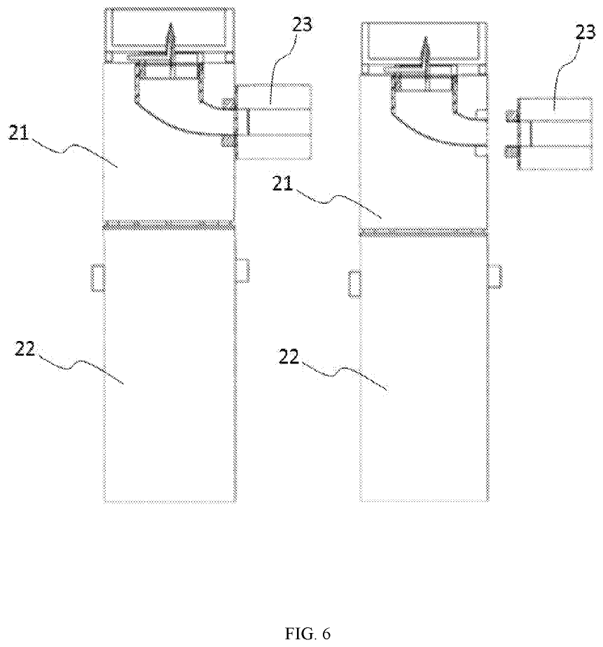

Specifically, as shown in FIG. 5, the mounting part 13 is fixedly connected to the bottom of the support 10, and the mounting part 13 is formed with a cavity; the atomizing device 2 is provided with the fitting part 214 connected to the mounting part 13 in a fitting manner; a position-limiting part 113 is fixedly connected to the puncture device 11, and the position-limiting part 113 can be fixedly connected to the fitting part 214; if the mounting part 13 is connected to the fitting part 214 in a fitting manner, the puncture device 11 can slide upwards relative to the support 10. Before use, a puncture part is positioned in the cavity of the mounting part 13; after the mounting part 13 is connected to the fitting part 214 in a fitting manner, the puncture device 11 enters the accommodating groove 101. Like Example 1, in this example, the mounting part 13 is also arranged as a plug with an external thread formed on the outer surface; the fitting part 214 is arranged as a socket, and the socket is a groove provided with an internal thread matching the external thread on the groove wall surface; the connecting device 1 is detachably connected to the atomizing device 2 through the external thread and the internal thread.

More specifically, the position-limiting part 113 has an inverted T-shaped structure, and comprises a horizontal rod 1131 extending along a horizontal direction and a vertical rod 1132 extending along a vertical direction; a lower-end portion of the vertical rod 1132 is fixedly connected to a middle portion of the horizontal rod 1131, and an upper-end portion of the vertical rod 1132 is fixedly connected to a lower end portion of the puncture device 11; the vertical rod 1132 is slidably connected to the mounting part 13, and the horizontal rod 1131 is detachably connected to the fitting part 214; when the horizontal rod 1131 is connected to the fitting part 214 in a fitting manner, the position-limiting part 113 can slide upwards relative to the support 10 to drive the puncture device 11 to slide upwards relative to the support 10. In this example, the horizontal rod 1131 is positioned below the mounting part 13, and can be slidably connected to the socket of the atomizing device 2; a position close to the bottom of the socket is provided with a position-limiting device, such that the horizontal rod 1131 cannot move downwards continuously; the vertical rod 1132 with the T-shaped structure passes through the bottom of the mounting part 13 and extends upwards to the liquid channel 111; the vertical rod 1132 is of a hollow structure and communicates with the liquid channel 111; the upper-end portion of the vertical rod 1132 is provided with an internal thread, a lower end of the liquid channel 111 of the puncture device 11 is provided with an external thread matching the internal thread of the vertical rod 1132, and the liquid channel 111 and the vertical rod 1132 are connected through threads. Of course, in other examples, if the hardness of the liquid channel 111 is enough, the liquid channel 111 can extend downwards to the horizontal rod 1131, such that when the support 10 is screwed into the socket, the puncture device 11 can be fixed.

In this example, the outer edge of the horizontal rod 1131 is an elastic material; the size of the horizontal rod 1131 is identical to that of the socket groove, and the horizontal rod can be directly inserted into the socket groove; the friction between the horizontal rod 1131 and the side wall of the socket groove can be increased when the plug is rotating, such that the puncture device 11 does not rotate along with the rotation of the plug; the support 10 moves downwards relative to the puncture device 11 and drives the vial 3 to move downwards; the elastic material used has medicinal liquid compatibility and biocompatibility.

During clinical use, the atomizing body 21 after being cleaned and disinfected is first in butt joint with the main unit 22 to form the atomizing main unit 22, at this time, the electrode probe 216 is in a point-to-point connection to the motor connector 223 to form a closed-loop circuit, and the atomizing sheet 212 is connected to a power supply of the main unit 22, such that the atomization process can be started at any time; then, the plastic part on the lid of the vial containing the medicinal liquid is removed to expose the rubber stopper, and the opening of the vial is mounted in the support 10 of the connecting device 1 in an inverted manner; the plug of the connecting device 1 is aligned with and inserted into the groove of the socket on the atomizing body 21, and the position-limiting part 113 at the bottom of the puncture device 11 is inserted into a position closed to the bottom of the socket groove and fixed thereto; the support 10 is rotated to screw the connecting device 1 into the socket in a rotating manner, and the support 10 moves downwards relative to the puncture device 11 and drives the vial 3 to move downwards along with the gradual insertion of the plug into the groove; then, the puncture device 11 punctures the rubber stopper, and the medicinal liquid flows from the liquid channel 111 of the puncture device 11 to the atomizing sheet 212 along the medicinal liquid channel 211 through the socket; finally, the power switch is pressed or turned on, and the power drives the grid type atomizing sheet 212 to vibrate, such that the high-efficiency atomization of the medicinal liquid is achieved; the protective sleeve 14 is not required to be additionally arranged in this example, which is more convenient to operate.

### Example 4

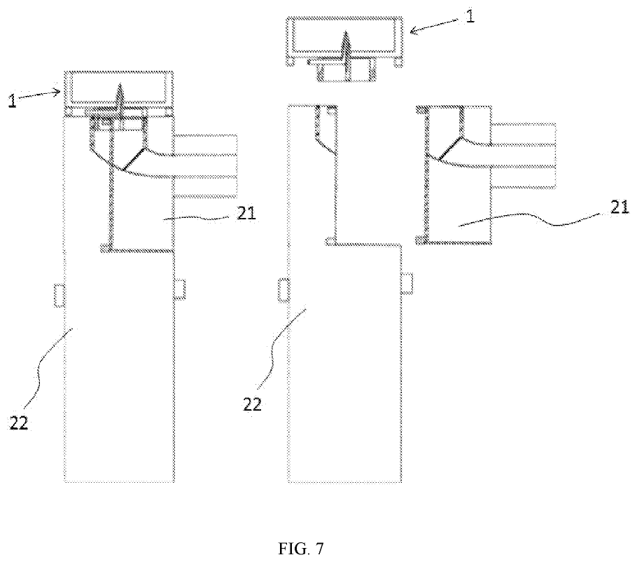

As shown in FIG. 6, this example provides an assembly method for a portable combined inhalation device suitable for vials.

Specifically, the atomizing device 2 of this example comprises the atomizing body 21 provided with the medicinal liquid channel 211, the atomizing sheet assembly 23 detachably connected to the atomizing body 21, and the main unit 22 detachably connected to the atomizing body 21; the atomizing sheet assembly 23 comprises a medicinal liquid outflow channel communicating with the medicinal liquid channel 211 of the atomizing body 21, and the medicinal liquid outflow channel is provided with the atomizing sheet 212 in an intercepting manner; when the atomizing sheet 212 needs to be cleaned, the atomizing sheet assembly 23 is directly detached for washing. In this example, the atomizing body 21 can be in the butt joint by a slot, a clasp, or a magnetic attraction. In this example, the atomizing sheet 212 assembly and the atomizing body 21 are in butt joint by a clasp, and the connection manner between the connecting device 1 and the atomizing body 21 and the connection manner between the atomizing body 21 and the main unit 22 are the same as those in Example 1.

### Example 5

As shown in FIG. 7, this example provides another assembly method for a portable combined inhalation device suitable for vials.

In this example, the socket and the medicinal liquid channel 211 are formed by splicing two detachable groove bodies.

Specifically, the main unit 22 and the atomizing body 21 of the atomizing device 2 are combined to form the socket, and the connecting device 1 is detachably connected to the socket through the plug; the main unit 22 and the atomizing body 21 are also combined to form the medicinal liquid channel 211, the medicinal liquid channel 211 communicates with the socket, and the atomizing sheet 212 is arranged in the medicinal liquid channel 211 in the atomizing body 21 in an intercepting manner; when the atomizing sheet 212 needs to be cleaned, the atomizing body 21 is directly detached for washing.

Specifically, the atomizing body 21 and the main unit 22 are connected in a snap-fit manner; preferably, the main unit 22 is provided with the unlock button 222, and after the unlock button 222 is operated, the connector 215 can be separated from the interface 224, so that the main unit 22 and the atomizing body 21 can be quickly detached.

In summary, the present application adopts the aforementioned embodiments, and has the following features:

Firstly, the connecting device can be adapted to vials 3 with various standard sizes without opening additional mold and non-standard filling lines, so that the production cost of the medicament is controlled; the puncture device 11 of the connecting device 1 punctures the vial 3, to avoid the waste and contamination of the medicinal liquid caused by transferring the medicinal liquid to the dedicated bottle of the atomizing device 2, and the medicinal liquid can be rapidly atomized by flowing to the atomizing sheet 212.

Secondly, the manner that a plurality of parts are detachably assembled is adopted, such that the atomizing sheet 212 assembly and the main unit 22 can be cleaned and disinfected conveniently and can be reused many times, and moreover, the probability of cross-contamination or infection of medicinal liquids caused by the atomization of different medicaments can be reduced.

In addition, in each of the aforementioned examples, after the atomization, inhalation, and administration are finished, the connecting device 1 is rotated and unscrewed from the atomizing main unit 22, taken out, and then classified and recycled as medical waste; meanwhile, a snap-fitting button on the main unit 22 is pressed, and the atomizing body 21 or the atomizing sheet assembly 23 is taken down; after soaking in high-temperature boiled water for 2 min, the atomizing body or the atomizing sheet assembly are wiped dry with medicinal cotton, put into a sterile plastic bag, and stored for later use; alternatively, after a disinfectant is added to the screw groove to disinfect the atomizing sheet 212 and the inner wall of the screw groove, the outer surface of each part of the device is cleaned and disinfected, and then these parts are independently packaged, sealed, and stored for later use. The present invention has been described in detail above, but the present invention is not limited to the aforementioned examples. All equivalent changes and modifications made according to the spirit of the present invention should fall within the protection scope of the present invention.

1. A portable combined inhalation device suitable for vials, wherein the inhalation device comprises:
an atomizing device (2); and
a connecting device (1) detachably connected to the atomizing device (2), the connecting device (1) comprising:
a support (10) on which an accommodating groove (101) capable of accommodating a vial is formed, and
a puncture device (11) that is arranged on the support (10) and can puncture a rubber stopper of the vial, wherein the puncture device (11) comprises a liquid channel (111) and a gas channel (112); if the vial, the connecting device (1), and the atomizing device (2) are connected, one end of the liquid channel (111) communicates with the vial, and the other end communicates with the atomizing device (2); one end of the gas channel (112) communicates with the vial, and the other end communicates with air.

2. The portable combined inhalation device suitable for vials according to claim 1, wherein the puncture device (11) has a length of 2-30 mm, and the liquid channel (111) and the gas channel (112) each independently have a diameter of 0.4-7 mm.

3. The portable combined inhalation device suitable for vials according to claim 1, wherein an upper end of the liquid channel (111) and an upper end of the gas channel (112) are integrally formed as a sharp end of the puncture device (11), or the liquid channel (111) and the gas channel (112) are separately arranged, and the upper end of the liquid channel (111) and the upper end of the gas channel (112) form a sharp end, respectively.

4. The portable combined inhalation device suitable for vials according to claim 1, wherein the puncture device (11) is fixedly connected to the support (10), extends upwards from the bottom of the support (10), and is positioned in the accommodating groove (101).

5. The portable combined inhalation device suitable for vials according to claim 4, wherein the puncture device (11) is sleeved with a detachable protective sleeve (14).

6. The portable combined inhalation device suitable for vials according to claim 1, wherein the puncture device (11) is slidably connected to the support (10), and the puncture device (11) is driven to pass through the bottom of the support (10) and protrude upwards into the accommodating groove (101) in the process of connecting the connecting device (1) to the atomizing device (2).

7. The portable combined inhalation device suitable for vials according to claim 6, wherein a mounting part (13) is fixedly connected to the bottom of the support (10), and the mounting part (13) is formed with a cavity; the atomizing device (2) is provided with a fitting part (214) fittingly connected to the mounting part (13); a position-limiting part (113) is fixedly connected to the puncture device (11), and the position-limiting part (113) can be fixedly connected to the fitting part (214); when the mounting part (13) is fittingly connected to the fitting part (214), the puncture device (11) can slide upwards relative to the support (10).

8. The portable combined inhalation device suitable for vials according to claim 7, wherein the position-limiting part (113) comprises a horizontal rod (1131) extending along a horizontal direction and a vertical rod (1132) extending along a vertical direction; a lower end portion of the vertical rod (1132) is fixedly connected to a middle portion of the horizontal rod (1131), and an upper end portion of the vertical rod (1132) is fixedly connected to a lower end portion of the puncture device (11); the vertical rod (1132) is slidably connected to the mounting part (13), and the horizontal rod (1131) is detachably connected to the fitting part (214); when the horizontal rod (1131) is fittingly connected to the fitting part (214), the position-limiting part (113) can slide upwards relative to the support (10) to drive the puncture device (11) to slide upwards relative to the support (10).

9. The portable combined inhalation device suitable for vials according to claim 8, wherein the vertical rod (1132) is of a hollow structure; if the connecting device (1) is connected to the atomizing device (2), one end of the vertical rod (1132) communicates with the liquid channel (111), and the other end communicates with the atomizing device (2).

10. The portable combined inhalation device suitable for vials according to claim 1, wherein an upper-end opening of the gas channel (112) is higher than an upper-end opening of the liquid channel (111).

11. The portable combined inhalation device suitable for vials according to claim 1, wherein if the connecting device (1) is connected to the atomizing device (2), a gap facilitating the communication of the gas channel (112) with the air exists between the bottom of the support (10) and the atomizing device (2).

12. The portable combined inhalation device suitable for vials according to claim 11, wherein the bottom of the support (10) is provided with a position-limiting block (12); if the connecting device (1) is connected to the atomizing device (2), a lower end portion of the position-limiting block (12) abuts against the atomizing device (2).

13. The portable combined inhalation device suitable for vials according to claim 1, wherein the connecting device (1) is detachably connected to the atomizing device (2) through threads.

14. The portable combined inhalation device suitable for vials according to claim 1, wherein the atomizing device (2) comprises a medicinal liquid channel (211), an atomizing sheet (212) arranged in the medicinal liquid channel (211) in an intercepting manner, and a motor; when the connecting device (1) is connected to the atomizing device (2), the liquid channel (111) communicates with the medicinal liquid channel (211).

15. The portable combined inhalation device suitable for vials according to claim 1, wherein the atomizing device (2) is detachably assembled from a plurality of parts.
